(19) United States
(12) Patent Application Publication
(10) Pub. No.: US 2010/0168583 A1
(43) Pub. Date: Jul. 1, 2010

(54) ENHANCED ULTRASOUND IMAGING PROBES USING FLEXURE MODE PIEZOELECTRIC TRANSDUCERS

(75) Inventors: David Dausch, Raleigh, NC (US); Olaf Von Ramm, Efland, NC (US); John Castellucci, Durham, NC (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: Research Triangle Institute, Research Triangle Park, NC (US)

(21) Appl. No.: 12/447,915

(22) PCT Filed: Nov. 3, 2006
(86) PCT No.: PCT/US06/43061

§ 371 Date: Feb. 19, 2010

(51) Int. Cl. A61B 8/14

(52) U.S. Cl. 600/466

(57) ABSTRACT

A method of generating an enhanced receive signal from a piezoelectric ultrasound transducer is described. The method comprises providing a piezoelectric ultrasound transducer comprising a piezoelectric element operable in flexural mode, receiving a acoustic signal by the piezoelectric element, applying a DC bias to the piezoelectric element prior to receiving the acoustic signal and/or concurrently with receiving the acoustic signal, and generating an enhanced receive signal from the piezoelectric element as a result of receiving the acoustic signal by the piezoelectric element. pMUT-based imaging probes using the above method are also described.

### FIELD

This invention relates to methods of generating enhanced flexure mode signals by piezoelectric transducers and ultrasound imaging probes using the same.

### BACKGROUND

Ultrasonic transducers are particularly useful for non-invasive as well as in vivo medical diagnostic imaging. Conventional ultrasonic transducers are typically fabricated from piezoelectric ceramic materials, such as lead zirconate titanate (PZT) or PZT-polymer composites, with the transducer material being diced or laser cut to form a plurality of individual elements arranged in one-dimensional or two-dimensional arrays. Acoustic lenses, matching layers, backing layers, and electrical interconnects (e.g., flex cable, metal pins/wires) are typically attached to each transducer element to form a transducer assembly or probe. The probe is then connected to control circuitry using a wire harness or cable, where the cable contains individual wires to drive and receive signals from each individual element. An important aim of ongoing research in ultrasonic transducer technology is increasing transducer performance and integrability with control circuitry while decreasing transducer size, power consumption and signal loss due to the cabling. These factors are particularly important for two-dimensional arrays required for three-dimensional ultrasound imaging.

Miniaturization of transducer arrays is particularly important for catheter-based 2D array transducers. A significant challenge is the complexity, cost of manufacture and limited performance of conventional 2D transducer arrays. Commercial 2D transducer probes are typically limited to arrays with element pitch of 200 to 300 μm and operating frequencies of <5 MHz. The small size of these elements drastically reduces the element capacitance to <10 pF which produces high source impedance and presents significant challenges with electrical impedance match to the system electronics. Furthermore, producing forward-looking 2D arrays for catheter-based intravascular (IVUS) or intracardiac (ICE) imaging probes has not been achieved commercially. With catheter size of 6 or 7 French or less, the transducer array should be less than 2 mm in diameter. For adequate resolution, a frequency of 10 MHz or greater should be used, which yields a wavelength of 150 μM in tissue. Because element pitch should be less than the wavelength for adequate imaging performance, element pitch of 100 μm or less is desired. Additionally, higher frequency operation requires a thinner piezoelectric layer in the transducers. To date, conventional transducer arrays have not met these requirements with a low cost, manufacturable process and adequate imaging performance.

The production of miniaturized transducers with adequate performance can be facilitated by micromachining techniques. The medical devices field, for example, has benefited from microelectromechanical systems (MEMS) technology. MEMS technology allows medical devices or their components to be manufactured with significant size reduction. Piezoelectric micromachined ultrasonic transducers (pMUTs) are one such MEMS-based transducer technology. pMUTs generate or transmit ultrasonic energy through application of AC voltage to a piezoelectric material suspended membrane causing it to undergo flexural mode resonance. This causes flextensional motion of the membrane to generate acoustic transmit output from the device. Received ultrasonic energy is transformed by the pMUT, with the ultrasonic energy generating a piezoelectric voltage (“receive signal”) due to flexural mode resonance vibrations of the microfabricated membrane.

The benefits of micromachined pMUT devices compared with conventional ceramic-based transducers include: ease and scalability of manufacture, especially for smaller, higher density 2D arrays; simpler integration and interconnection for 2D arrays; more flexibility in transducer design for wider operating frequency range; higher element capacitance for lower source impedance and better match with electronics. 2D arrays are needed for real-time 3D imaging systems, and ceramic transducers are quickly reaching their manufacturability limit for insertion into smaller catheter probes (2-3 mm diameter or smaller). Another micromachining approach is capacitive micromachined ultrasound transducers (cMUTs), consisting of surface micromachined membranes on a substrate that are actuated electrostatically by applying appropriate DC and AC voltage signals to the membrane electrodes. However, these devices require multiple elements connected in parallel to provide sufficient acoustic output, thus limiting the performance for 2D arrays with very small element size. Sizeable amplification (typically 60 dB) is required in order to obtain an ultrasound signal with cMUTs.

There are functional and structural differences between cMUT and pMUT devices. Because pMUTs have a higher energy transduction mechanism (i.e., the piezoelectric layer), the piezoelectric elements generally have higher ultrasonic power capability than cMUTs. 2D array pMUT elements with 75 micron width can generate acoustic power output of 1 to 5 MPa at a frequency of 8 MHz. Conventional transducer arrays can generate >1 MPa acoustic pressure, but require much larger element size and operate at lower frequency. Typical acoustic output for cMUT 2D array elements is much less than 1 MPa. Elements in pMUT arrays also have higher capacitance (on the order of 100-1,000 pF) than conventional transducer arrays and cMUTs, producing lower source impedance and better impedance match to the cabling and electronics. Conventional transducer arrays elements have capacitance of <10 pF, and cMUT elements have capacitance of <1 pF.

pMUTs typically operate with lower voltages than conventional transducers and cMUTs. Depending on the thickness of the ceramic plate, conventional transducers can require high voltage bipolar signals (>100 V peak-to-peak) to generate acoustic energy. cMUTs require a large (>100V) DC voltage to control the membrane gap distance in addition to an AC signal (typically tens of V peak-to-peak) to vibrate the membrane. pMUTs require lower AC voltages (typically 30 V peak-to-peak bipolar signal) to acuate the piezoelectric vibration for transmitting acoustic energy, and the received ultrasonic energy causes flexural mode resonance generating the receive signals without the need for applied voltage.

Micromachined ultrasound transducers provide miniaturized devices that may be directly integrated with control circuitry. For example, cMUTs have been integrated with control circuitry with through-wafer via connections made by etching vias in a silicon wafer, coating the wafer with a thermal silicon dioxide for insulating regions and with polysilicon for electrical contacts, and then building up the cMUT membrane elements on the top surface of the wafer. Metal pads and solder bumps may be deposited on the bottom surface of the wafer in order to solder the cMUT chip to semiconductor device circuitry.

One disadvantage of such a cMUT device, however, is that relatively high resistivity polysilicon, compared to metals, is used as the conductive material in the vias because of processing limitations inherent in the cMUT architecture. Because of the already very low signal strength generated by cMUTs in the receive mode, the signal to noise ratio may be problematic during operation of the cMUT with polysilicon vias. Also, the low capacitance of cMUT elements produces high impedance, and therefore impedance mismatch with the electronics and cabling are greater, which contributes to increased signal loss and noise. High resistance in the through-wafer vias further exacerbates the high element impedance problem. In addition, significant resistance in the vias will cause more power consumption and heat generation during operation when applying drive signals to cMUTs for transmit.

Another disadvantage of the cMUT device with polysilicon through-wafer interconnects is the processing temperature of forming the thermal silicon dioxide insulator and the polysilicon conductor. Processing temperatures for these steps are relatively high (600-1000° C.), thus creating thermal budget issues for the rest of the device. Because of these processing temperatures, the cMUT elements must be formed after the through-wafer vias are formed, and this sequence creates difficult processing issues when trying to perform surface micromachining on a substrate with existing etched holes through the wafer.

Conventional transducer arrays may be integrated directly with control circuitry. However this typically requires solder bumping which is a relatively high temperature process (approximately 300° C.), and high density integration is not feasible due to the large size of the array elements (200 to 300 micron pitch at a minimum).

Thus, pMUT devices offer functional and fabrication advantages over conventional ultrasound transducers and cMUTs. Intravascular imaging and interventions are particular areas where miniaturized devices are desirable and where MEMS devices are attractive. An example of an application of a MEMS-type medical device is in imaging devices, such as intravascular ultrasound (IVUS) and intracardiac echo (ICE) imaging. IVUS devices, for example, provide real-time tomographic images of blood vessel cross sections, elucidating the true morphology of the lumen and transmural components of atherosclerotic arteries. Such devices, while offering great promise, are amenable to improvement in specific functionally dependent performance areas such as receive mode sensitivity.

### SUMMARY

In one embodiment, a method of generating an enhanced receive signal from a piezoelectric ultrasound transducer is provided. The method comprises providing a piezoelectric ultrasound transducer, the piezoelectric ultrasound transducer comprising a piezoelectric element operable in flexural mode and receiving acoustic energy by the piezoelectric element. The acoustic energy is convertible to an electrical voltage by flexural mode resonance of the piezoelectric element. The applied transmit voltage is a sine wave signal that includes an additional half-cycle of excitation. The resulting enhanced receive signal generated by the piezoelectric transducer is greater than the receive signal generated by the piezoelectric transducer for applied transmit voltage without the additional half-cycle excitation.

In yet another embodiment, a method of generating an enhanced receive signal from a piezoelectric ultrasound transducer is provided. The method comprises providing a piezoelectric ultrasound transducer, the piezoelectric ultrasound transducer comprising a piezoelectric element operable in flexural mode and receiving acoustic energy by the piezoelectric element. The acoustic energy is convertible to an electrical voltage by flexural mode resonance of the piezoelectric element. A DC bias is applied to the piezoelectric element prior to receiving the acoustic energy and/or concurrently with receiving the acoustic energy. An enhanced receive signal is generated from the piezoelectric transducer by converting the received acoustic energy to an electrical voltage by flexural mode resonance of the piezoelectric element. The enhanced receive signal generated by the piezoelectric transducer is greater than a receive signal generated by the piezoelectric transducer in the absence of applying a DC bias.

In another embodiment, a method of generating an enhanced receive signal from a piezoelectric ultrasound transducer is provided. The method comprises providing a piezoelectric ultrasound transducer (the piezoelectric ultrasound transducer comprising a piezoelectric element operable in flexural mode) and applying a sine wave bipolar transmit cycle pulse to the piezoelectric element to produce an acoustic signal providing an acoustic echo. The sine wave bipolar transmit cycle pulse has a maximum peak voltage. The acoustic echo is received by the piezoelectric element, which is convertible to an electrical voltage by flexural mode resonance of the piezoelectric element. A DC bias applied to the piezoelectric element prior to receiving the acoustic echo and/or concurrently with receiving the acoustic echo and an enhanced receive signal is generated from the piezoelectric transducer by converting the received acoustic echo to an electrical voltage by flexural mode resonance of the piezoelectric element. The enhanced receive signal generated by the piezoelectric transducer is greater than a receive signal generated by the piezoelectric transducer in the absence of applying a DC bias.

In yet another embodiment, an ultrasound imaging catheter is provided. The catheter comprises a substrate, a plurality of sidewalls defining a plurality of openings through the substrate and spaced-apart bottom electrodes on the substrate. Each spaced-apart bottom electrode spans one of the plurality of openings and spaced-apart piezoelectric elements on each of the bottom electrodes. Conformal conductive film on each of the sidewalls of the plurality of openings is in contact with one or more of the bottom electrodes and open cavities are maintained in each of the openings. Means for applying a DC bias to the piezoelectric transducer are included.

In yet another embodiment, an ultrasound imaging probe is provided. The catheter comprises a substrate, a plurality of sidewalls defining a plurality of openings partially through the substrate and spaced-apart piezoelectric elements on the substrate. Each spaced-apart piezoelectric element is positioned over one of the plurality of openings. Pairs of spaced-apart bottom electrodes on the substrate are in contact with each of the spaced-apart piezoelectric elements. Conformal conductive film on each of the sidewalls of the plurality of openings is in electrical interconnection with one or more of the bottom electrodes and open cavities are maintained in each of the openings.

In yet another embodiment, a method of generating an enhanced receive signal from a piezoelectric ultrasound transducer is provided. The method comprises providing a piezoelectric ultrasound transducer, the piezoelectric ultrasound transducer comprising a piezoelectric element operable in flexural mode and having a ferroelectric coercive voltage. A transmit voltage is applied to the piezoelectric transducer which is above the ferroelectric coercive voltage for the piezoelectric element. Acoustic energy is generated by the piezoelectric element providing an acoustic echo. An enhanced receive signal is generated from the piezoelectric transducer by converting the received acoustic echo to an electrical voltage by flexural mode resonance of the piezoelectric element. The resulting enhanced receive signal generated by the piezoelectric transducer is greater than the receive signal generated by the piezoelectric transducer for applied transmit voltage less than the coercive voltage.

### BRIEF DESCRIPTION OF THE FIGURES

FIG. 1 graphically represents an embodiment of the method of enhancing a receive signal.

FIGS. 2-3 illustrate a piezoelectric microfabricated ultrasonic transducer device wherein the transducer is attached to a semiconductor device according to an embodiment of the invention.

FIGS. 4-6 illustrate the formation of a piezoelectric microfabricated ultrasonic transducer device wherein the transducer is attached to a semiconductor device according to an embodiment of the invention.

FIG. 7 illustrates a piezoelectric microfabricated ultrasonic transducer device wherein the piezoelectric elements are formed on a doped silicon-on-insulator substrate.

FIG. 8 illustrates a piezoelectric microfabricated ultrasonic transducer device wherein the transducer is attached to a semiconductor device according to an embodiment of the invention.

FIGS. 9-15 illustrate an imaging catheter comprising a piezoelectric microfabricated ultrasonic transducer device according to an embodiment of the invention.

FIG. 16 illustrates an imaging probe embodiment.

### DETAILED DESCRIPTION

The embodiments disclosed herein relate to methods of enhancing the sensitivity of at least one piezoelectric element of an ultrasound flex mode transducer by applying a transmit voltage sine wave signal that is above the ferroelectric coercive field and/or contains an additional half-wave excitation in the sine wave signal. The embodiments further relate to methods of enhancing the sensitivity of a imaging device operating with an ultrasound flex mode transducer by applying a DC bias before and/or with the receive flexural mode resonance of the piezoelectric element of the ultrasound flex mode transducer. The embodiments further relate to methods of enhancing the sensitivity of a imaging device operating with an ultrasound flex mode transducer by applying a DC bias with the receive flexural mode resonance of at least one piezoelectric element of the ultrasound flex mode transducer. The embodiments herein further relate to improved silicon-on-insulator pMUT (SOI-pMUT) elements, their manufacture and use with methods enhancing their sensitivity by applying a transmit voltage above the coercive voltage, additional half-wave excitation, and/or DC bias with the receive flexural mode resonance of the SOI-pMUT elements. The embodiments herein further relate to imaging devices comprising flex mode transducer elements and methods of enhancing their sensitivity by applying a transmit voltage above the coercive voltage, additional half-wave excitation, and/or DC bias with the receive flexural mode resonance of the flex mode transducer elements. The embodiments herein described are generally applicable to medical ultrasound diagnostic imaging probes comprising flex mode transducers such as pMUTs.

The terms “microfabricated,” “micromachining” and “MEMS” are used interchangeably and generally refer to methods of manufacturing used in integrated circuit (IC) manufacture.

The terms “flexural mode,” “flexure mode,” “flex mode” and “flextensional mode” are used interchangeably and generally refer to expansion and contraction of a suspended piezoelectric membrane resulting in flex and/or vibration of the piezoelectric membrane.

As used herein, the term “flexural mode resonance” refers generally to excited axisymmetric resonant modes of flex mode transducer elements that generate ultrasound acoustic energy of specific frequencies or are caused by receipt of ultrasound acoustic energy of specific frequencies.

As used herein, the terms “ferroelectric coercive voltage,” “coercive voltage” and “coercive field” are used interchangably and refer to the voltage above which ferroelectric dipole switching of a piezoelectric material occurs. Coercive field may be in the range of 1 to 10 V/micron. For example, a piezoelectric membrane with a thickness of 1 micron typically has a coercive voltage of approximately 3 to 5 V.

A method for generating enhanced receive signals of a flex mode transducer is provided. The method comprises applying a DC bias during and/or prior to receive flexural mode resonance of a piezoelectric element. The method is generally applicable during pulse-echo operation of a flex mode transducer such as a pMUT. The method may be adapted to a flex mode transducer using vertically integrated pMUT array. The method may further be adapted to catheter-based imaging devices comprising a pMUT array and/or a vertically integrated pMUT array to enhance the receive signal during pulse-echo operation.

A method for generating enhanced receive signals of a flex mode transducer is provided. The method comprises applying a transmit voltage sine wave signal that is above the ferroelectric coercive voltage of the piezoelectric material. The method also comprises applying an additional half-wave excitation in the applied transmit sine wave signal. The method may be combined with applying a DC bias to the piezoelectric element prior to receiving the acoustic echo and/or concurrently with receiving the acoustic echo. The method is generally applicable to a flex mode transducer having a thickness-dependent coercive voltage.

Flexure mode operation presents a unique method for generating acoustic energy that is significantly different from methods used with conventional ultrasound transducers which typically operate with thickness mode vibration. Conventional transducers consist of pre-poled piezoelectric ceramic plates that operate below the coercive voltage to generate vibration in the thickness direction of the plate. Conventional transducers contain piezoelectric ceramic plates that are relatively thick (hundreds of microns thick), thus it is not practical to operate above the coercive voltage which would require transmit voltage signals of several hundred volts. Furthermore, operating above the coercive field would depole the ceramic and require repoling at high voltage (hundreds of volts) to achieve sufficient receive sensitivity.

pMUT devices may operate by applying a bipolar signal at voltage levels above the coercive field in order to induce 90° domain switching in the PZT thin film. The PZT film is very thin (one to several microns thick), thus operation above the coercive voltage can be achieved at relatively low operating voltage levels (tens of volts). Internal stress in piezoelectric thin films reduces the ferroelectric polarization of the piezoelectric material. The internal stresses in the piezoelectric thin films restrict the ferroelectric dipoles, which may result in non-ideal alignment of the ferroelectric dipoles in the absence of an applied voltage. By forcing alignment of the ferroelectric dipoles, some polarization recovery may be achieved by applying a voltage greater than the coercive voltage; however, when the voltage is removed, internal stresses reduce the alignment of the ferroelectric dipoles. Thus, pre-poling the films does not achieve the maximum dipole alignment, as is the case in conventional bulk ceramic piezoelectric transducers.

The methods herein described are in contrast to the typical operation of ultrasound transducers using a piezoelectric transducer (conventional or pMUT), which transmit with voltage below the ferroelectric coercive voltage. Transmitting with voltage above the coercive voltage forces the piezoelectric material to undergo ferroelectric 90° domain switching, thus maximizing the flexure of the membrane through flextensional motion. The method also describes applying an additional half-wave excitation in the sine wave signal to force preferred dipole alignment to enhance the pulse-echo receive sensitivity.

The methods herein described are also in contrast to the typical operation of ultrasound transducers using a piezoelectric transducer (conventional or pMUT), which receives echo signals in the absence of an applied voltage. The method for improving the receive signal of a flex mode piezoelectric transducer includes applying a DC bias voltage before and/or during receipt of the acoustic signal by the piezoelectric element. Application of a DC bias before and/or during flexural mode resonance of a piezoelectric element of a flex mode transducer increases the receive signal (e.g., output current) of the piezoelectric element. When receiving acoustic echo signals, the piezoelectric layer in a pMUT is not necessarily poled to its maximum extent. One cause of this reduced poling is that the transmit voltage itself may depole all or part of the piezoelectric layer. Thus, the application of DC bias enhances the dipole alignment and resulting pulse echo receive signal.

The method of generating enhanced receive signals is discuss below with reference to a pMUT of particular design but the method is generally applicable to any microfabricated piezoelectric elements and piezoelectric ultrasound elements operating in flexural mode.

The method may be performed, by way of example, as follows. Acoustic energy directed towards a pMUT element is provided. The acoustic energy may be reflected energy generated from the same piezoelectric element that will receive the acoustic energy, reflected energy from a different piezoelectric element of an array or reflected energy from another source. By way of example, reflected energy from the piezoelectric element as an acoustic echo (pulse-echo) will be discussed.

In one aspect of the method, a bipolar transmit voltage is applied that is above the coercive voltage of the piezoelectric material. This high electric field level enhances the ferroelectric 90° domain switching in the piezoelectric layer which increases the vibration amplitude of the membrane. This results in higher acoustic energy output from the membrane; therefore, higher pulse echo signal is received due to the higher transmit energy output. The pulse echo signal can also be enhanced by applying an additional half-cycle excitation to the piezoelectric element in the transmit signal. Typical transmit voltage pulses contain one, two or three full-cycle pulses. Increasing the number of pulses generally increases the transmit output of the transducer at the expense of resolution. It is an aspect of this method to apply an additional half-cycle excitation, i.e., 1.5, 2.5, or 3.5 cycles, to increase the sensitivity of the pMUT element without significantly sacrificing resolution capability compared to 1, 2 or 3 cycle pulses. It has been shown that pMUT elements produce higher pulse echo receive signals as a result of applying the additional half-cycle transmit excitation compared to full cycle excitation. This is due to enhanced dipole alignment in the piezoelectric layer of the pMUT element.

In another aspect of the method, before the acoustic echo reaches the transducer, a DC bias may be applied to the piezoelectric element and then held while the piezoelectric element is in flexural resonance mode from the received echo. The DC bias improves the dipole alignment in the piezoelectric material and thus increases the receive signal generated by the membrane. Because the dipole alignment is improved, higher piezoelectric current is generated as a result of received acoustic wave producing mechanical vibration in the membrane. The DC bias can also be applied to an array of piezoelectric elements, wherein the applied DC bias may be the same for all elements or may vary from element to element. pMUT elements can have some variability in their pulse echo receive characteristics; therefore, applying a calibrated DC bias to each element in the array during receive flexure mode resonance can also improve the receive signal uniformity across the array for a given acoustic pressure to enhance the resulting ultrasound image quality.

In another aspect of the method, a bipolar transmit voltage may be applied to the pMUT to emit acoustic energy. The acoustic energy is reflected from the target as an acoustic echo, and returns toward the pMUT. Before the acoustic signal reaches the transducer, a DC bias pulse is applied to the transducer prior to the receive flexural resonance mode and removed prior to the receive flexural resonance mode of the piezoelectric elements. Without being limited by theory, it is generally believed that the DC bias pulse temporarily improves the dipole alignment and that upon removal of the DC bias pulse the dipole alignment does not immediately revert to its internally stressed state. Thus, the piezoelectric current output that results from the receive flexural resonance mode is increased due to residual polarization from the dipole alignment. Piezoelectric output may be lower than in the previously mentioned aspect of the method because the dipole alignment is not maximized during receive flexural resonance mode. However, this method may obviate the requirement of additional signal conditioning circuitry. Moreover, as the pulse may be of a shorter duration than the previously described aspect of the method wherein the DC bias is held while the piezoelectric element is in flexural resonance mode from the received echo, overall power consumption may be reduced. Because the prior transmit voltage cycle may depole the piezoelectric material, this method provides enhanced domain alignment of a known polarity (in the direction of the DC bias polarity) to produce an enhanced receive signal.

In another aspect of the method, a bipolar transmit voltage is applied to the pMUT to emit acoustic energy. The bipolar transmit voltage is stopped at maximum peak voltage. The bipolar transmit voltage may be a sine wave transmit cycle pulse or other periodic pulse. The acoustic energy is reflected from the target as an acoustic echo, and returns toward the pMUT. By stopping the voltage of the transmit cycle at peak voltage, retention of dipole alignment may be obtained, which may increase the piezoelectric current generated by receive flexural resonance mode of the piezoelectic elements from the echo signal. The bipolar transmit voltage may be stopped at a voltage between maximum voltage and zero voltage during the transmit cycle. This aspect of the method may be combined with other the other aspects of the method to enhance the receive signal from the pMUT.

In another aspect of the method, a bipolar transmit voltage is applied to the pMUT to emit acoustic energy. The bipolar transmit voltage is stopped at maximum peak voltage. The bipolar transmit voltage may be a sine wave transmit cycle pulse or other periodic pulse. The acoustic energy is reflected from the target as an acoustic echo, and returns toward the pMUT. Before the acoustic signal reaches the transducer, a DC bias with opposite sign of that of the transmit peak voltage is applied to the transducer and then held during receive flexural resonance mode of the piezoelectic elements. Without being held by theory, it is believed that this aspect of the method forces the ferroelectric dipoles to switch during receive flexural resonance mode of the piezoelectic elements from the receive echo. Dipole switching may generate additional piezoelectric current that may amplify the signal generated by the receive echo. The bipolar transmit voltage may be stopped at a voltage between maximum voltage and zero voltage during the transmit cycle provided that a DC bias with opposite sign to that of the stopped transmit cycle voltage is used. Combinations of the above aspects are included in the scope of the method.

The timing of the application of the DC bias may be calculated based on the frequency of the pMUT device and the target depth in the imaging arena. The DC bias may be adjusted or chosen to account for internal stresses of the piezoelectric membrane layer. The DC bias may be swept from 0 to positive or 0 to negative. As the transmit cycle pulse is of the order of nanoseconds, whereas the echo return is typically on the order of microseconds, the DC bias duration may be pulsed, constantly applied, applied in other fashions or applied in combinations with aspects of the method described herein such that the receive signal is enhanced.

Signal conditioning electronic circuitry may be implemented to separate the DC bias signal from the generated piezoelectric receive signal and/or to reduce or prevent noise in the receive signal. Signal conditioning circuits may be integrated directly adjacent to the pMUT substrate or may be integrated in vertically stacked ASIC devices. Integration of ASIC devices using through-wafer interconnect schemes may be as described in co-pending U.S. patent application Ser. No. 11/068,776, incorporated herein by reference in its entirety. Signal conditioning circuits integrated with the pMUT substrate may reduce noise in the receive signal. Signal conditioning may be applied to amplify the receive signal. Multiple IC's may be stacked with the pMUT using through-wafer interconnect processing such that signal conditioning and amplification circuitry is integrated in close proximity with the pMUT device for maximizing signal and/or reducing noise which may result from application of DC bias. Signal conditioning may be performed remotely. Means for applying the DC bias to the piezoelectric elements include a pair of electrically conductive contacts driven by and in electrical communication with a potential source. The electrical communication includes wires, flex cabling, and the like. Potential sources include a battery, AC or source/drain and the like. The electrically conductive contacts, in communication with a potential source, may be connected to the piezoelectric elements such that an active electrical circuit is created and controlled. Such electrically conductive contacts may be in series or parallel with the elements. Means and equivalents thereof include additional circuitry and/or electronic components designed to control the DC bias concurrently with transmit and receiving signals as is within the ordinary skill of the skilled artisan, such as with filtering or low noise amplifiers.

Application of the above method of generating enhanced receive signal may be integrated with the pMUT and silicon-on-insulator (SOI) substrate pMUT devices (SOI-pMUT) and/or vertically stacked ASIC-pMUT devices as disclosed in co-pending application U.S. patent application Ser. No. 11/068,776, for example, as described below.

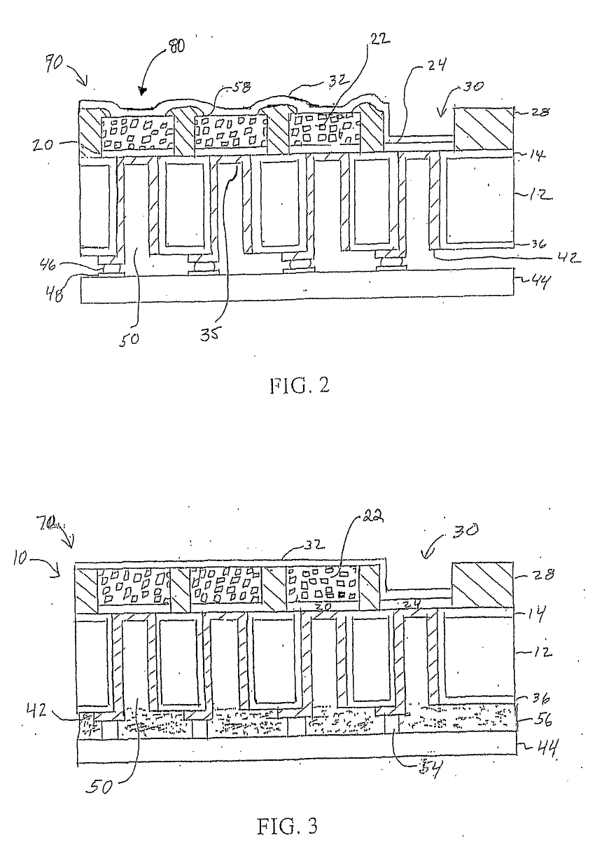

Referring to FIG. 2, a pMUT device structure 80 is shown connected to a semiconductor device 44 to form a vertically integrated pMUT device 90. By way of example, the connection is made through solder bumps 46 connecting the conformal conductive layer 42 to solder pads 48 on the semiconductor device 44.

Top electrode 32 and bottom electrode 20 sandwich piezoelectric array elements 22 separated by second dielectric 28 which overlaps edges 58 of elements 22. Bottom electrodes 20 are isolated by first dielectric layer 14 which is etched away during subsequent formation of air-backed cavities 50 in back side of substrate 12. Air back cavities 50 have sidewalls coated with conformal insulating film 36 and conformal conductive film 42 providing thru-wafer via interconnect of the semiconductor device 44 with the piezoelectric array elements 22. The patterned through-wafer interconnects 42 provide direct electrical connection from the piezoelectric membranes 35 to the semiconductor device 44 and ground pad 24 in opening 30. The air-backed cavities 50 provide optimum acoustic performance. The air-backed cavity 50 allows greater vibration in the piezoelectric membrane 35 with minimal acoustic leakage compared to surface microfabricated MUTs.

Vertically integrated pMUT device 90 including second dielectric film 28 on the top edges of the patterned piezoelectric layer 58 provides improved electrical isolation of the two electrodes 32, 20 connected to the piezoelectric elements 22. This embodiment helps account for any photolithography misalignment which could inadvertently cause a gap between the polymer dielectric 28 and piezoelectric element 22 edges causing the top electrode 32 to short to the bottom electrode 20. The second dielectric film 28 also eliminates the need for any planarization processes that might be required in other embodiments. This embodiment further provides a method of forming a size or shape of the top electrode 32 that is different from the size and shape of the patterned piezoelectric elements 22. If thick enough (on the order of the piezoelectric thickness), the second dielectric film 28 with much lower dielectric constant than the piezoelectric elements 22 causes the voltage applied to the pMUT 90 device to primarily drop only across the dielectric, thus electrically isolating the portion of the piezoelectric layer 58 that is covered with the dielectric. The effective shape of the piezoelectric element 22 with regard to the applied voltage is only the portion of the piezoelectric elements 22 that is not covered with the dielectric. For example, if it is desired only to electrically activate 50% of the total piezoelectric geometrical area, then polymer dielectric 28 may physically cover and electrically isolate the remaining 50% of the piezoelectric area and prevent it from being activated. Also, if a complex electrode pattern is desired such as an interdigitated structure, a polymer dielectric may be used for the second dielectric layer 28 and may be patterned to provide the interdigitated structure. This is important for certain embodiments wherein the top electrode 32 is a continuous ground electrode across the entire pMUT array. Simpler processing is provided by creating the electrically active area by patterning the polymer dielectric 28, thus the active area assumes the shape of the top electrode area contacting the piezoelectric element 22, rather than patterning the bottom electrode 20 and a piezoelectric film.

Vibrational energy from surface microfabricated membranes may be dissipated into the bulk silicon substrate which resides directly below the membrane thus limiting the ultrasonic transmit output and receive sensitivity. The air backed cavity 50 of the present invention reduces or eliminates this energy dissipation since the vibrating membrane 35 does not reside directly on or over the bulk substrate 12.

The semiconductor device 44 may be any semiconductor device known in the art, including a wide variety of electronic devices, such as flip-chip package assemblies, transistors, capacitors, microprocessors, random access memories, multiplexers, voltage/current amplifiers, high voltage drivers, etc. In general, semiconductor devices refer to any electrical device comprising semiconductors. By way of example, the semiconductor device 44 is a complementary metal oxide semiconductor chip (CMOS) chip.

Because each piezoelectric element 22 is electrically isolated from adjacent piezoelectric elements 22, the individual elements may be separately driven in the transducer transmit mode. Additionally, receive signals may be measured from each piezoelectric membrane independently by the semiconductor device 44. Receive signals may be enhanced by the method of applying a DC bias for each or every piezoelectric element independently by the semiconductor device 44. Receive signal conditioning and DC bias circuitry may be integrated with semiconductor device 44.

An advantage of the formation of the through-wafer interconnects 42 is that separate wires, flex cable, etc., are not required to carry electrical transmit and receive signals between the membranes 35 and semiconductor device 44, as electrical connection is provided directly by the interconnects 42. This reduces the number of wires and size of the cabling required to connect the ultrasonic probe to a control unit. Furthermore, the shorter physical length of the through-wafer interconnects 42 (<1 mm) compared with conventional cable or wire harnesses (length on the order of meters) provides connections with lower resistance and shorter signal path which minimizes loss of the transducer receive signal and lowers the power required to drive the transducers for transmit.

The use of metal interconnects 42 and electrodes 20, 32 may provide a piezoelectric device with higher electrical conductivity and higher signal-to-noise ratio than devices using polysilicon interconnects and electrodes. In addition, the use of low temperature processes of depositing the conformal insulating layer 36 and conformal conductor 42 reduces the thermal budget of the device processing, thus limiting the damaging effects of excessive exposure to heat. This also allows the piezoelectric elements 22 to be formed before etching the through-wafer via holes 50 in the substrate, thus simplifying the overall processing.

When a pMUT device structure is attached directly to a semiconductor device substrate, there may be observed some reverberation of the pMUT elements as acoustic energy is reflected off of the semiconductor device substrate and directed back toward the piezoelectric membrane. The reverberation causes noise in the pMUT signal and reduces ultrasound image quality. Also the acoustic energy could affect semiconductor device operation by introducing noise in the circuit. By way of example, using an acoustic dampening polymer coating on the contacting surface of the semiconductor device, or at the base of the air-backed cavity of the pMUT device, acoustic energy reflected from the piezoelectric membrane may be attenuated. The acoustic dampening polymer layer preferably has a lower acoustic impedance and reflects less ultrasonic energy than a bare silicon surface of the semiconductor device with high acoustic impedance. By way of example, the acoustic dampening polymer layer may be also function as an adhesive for attachment of the pMUT device structure to a semiconductor device.

The thickness of piezoelectric elements 22 of the pMUT device may range from about 0.5 μm to about 100 μm. By way of example, the thickness of the piezoelectric elements 22 ranges from about 1 μm to about 10 μm.

The width or diameter of the piezoelectric elements 22 may range from about 10 μm to about 500 μm with center-to-center spacing from about 15 μm to about 1000 μm. By way of example, the width or diameter of the piezoelectric elements 22 may range from about 50 μm to about 300 μm with center-to-center spacing from about 75 μm to 450 μm for ultrasonic operation in the range of 1 to 20 MHz. Smaller elements of less than 50 μm may be patterned for higher frequency operation of >20 MHz. By way of example, multiple elements may be electrically connected together to provide higher ultrasonic energy output while still maintaining the high frequency of operation.

The thickness of the first dielectric film 14 may range from about 10 nm to about 10 μm. By way of example, the thickness of the conformal insulating film 36 ranges from about 10 nm to about 10 μm. The thickness of the bottom electrode 20, top electrode 32, and conformal conductive layer 42 ranges from about 20 nm to about 25 μm. The depth of the open cavity 50 may be range from about 10 μm to several millimeters.

In one embodiment, a pMUT device structure 10 is connected to the semiconductor device 44 through metal contacts 54 formed in the epoxy layer 56 on the semiconductor device 44 forming vertically integrated pMUT device 70, as illustrated in FIG. 3. The epoxy layer 56, in addition to functioning as an acoustic energy attenuator, may also function as an adhesive for adhering the pMUT device structure 10 to the semiconductor device 44. The epoxy layer 56 may be patterned using photolithographic and/or etching techniques, and metal contacts may be deposited by electroplating, sputtering, electron beam (e-beam) evaporation, CVD, or other deposition methods.

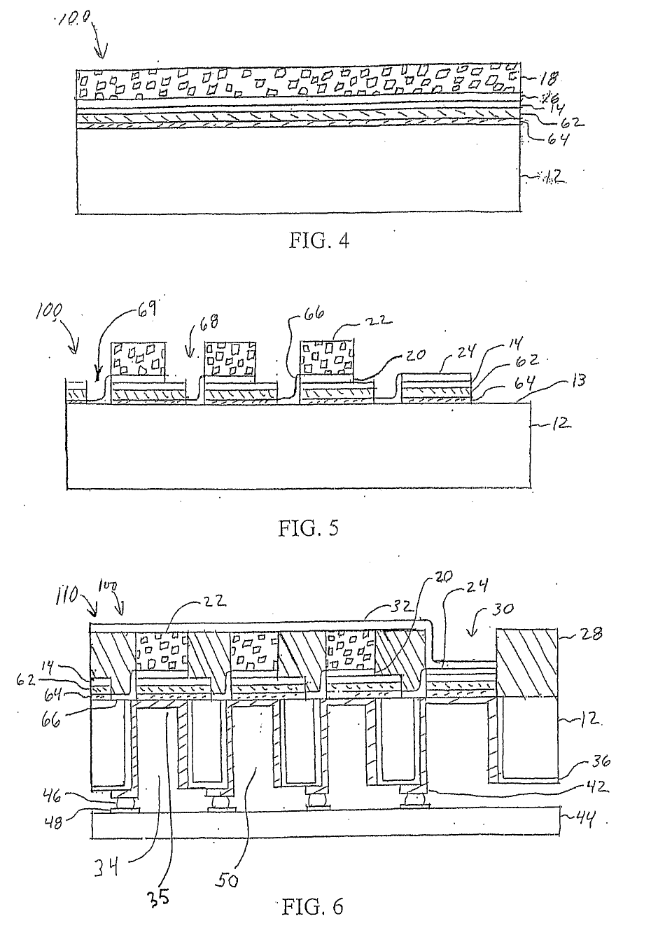

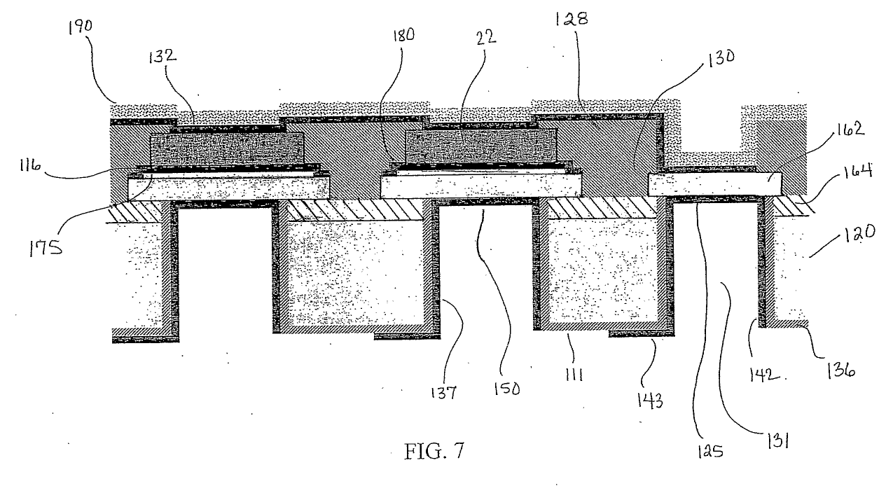

In certain embodiments, application of the above method of enhancing receive signal may be integrated with the pMUT fabricated with a silicon-on-insulator (SOI) substrate as the substrate as previously described in co-pending U.S. patent application Ser. No. 11/068,776, as shown in FIGS. 4-6, for example, as well as an improved SOI-pMUT device as described below with reference to FIG. 7.

As shown in FIG. 4, a substrate 12, such as a silicon wafer, is provided with a thin silicon layer 62 overlying a buried silicon dioxide layer 64 formed on the substrate 12. A first dielectric film 14 is formed overlying the silicon layer 62 and a bottom electrode layer 16 is formed overlying the first dielectric film. A layer of piezoelectric material 18 is formed overlying the bottom electrode layer 16 to provide a SOI pMUT device structure 100. At least one advantage of using the SOI substrate includes better control of the deep reactive ion etching (DRIE) using the buried oxide as the silicon substrate etch stop. The SOI also provides better control of the pMUT membrane 35 thickness for better control and uniformity of the resonance frequencies of the individual elements in an array, as the membrane thickness is defined by the thickness of the thin silicon layer of the SOI substrate 62. According to certain embodiments, the thin silicon layer 62 has thickness of about 200 nm to 50 μm, and the buried oxide layer 64 has thickness of about 200 nm to 1 μm. In other embodiments of the present invention, the thin silicon layer 62 has thickness of about 2 μm to 20 μm, and the buried oxide layer 64 has thickness of about 500 nm to 1 μm.

With reference to FIG. 5, the layer of piezoelectric material 18, bottom electrode layer 16, first dielectric film 14, silicon layer 62, and buried silicon oxide layer 64 are subsequently etched to provide separate piezoelectric elements 22 and a ground pad 24, and to expose the front side 13 of the substrate 12. The piezoelectric 18 and bottom electrode 16 layers are etched to form the pMUT element shape 22 separated by openings 68. The first dielectric 14, thin silicon 62, and buried oxide 64 layers are further etched to form spaced-apart vias 69 exposing the substrate 12. A conductive film 66 is deposited in the spaced-apart vias 69, as illustrated in FIG. 5, to provide electrical connection between the bottom electrode 20 and the through-wafer interconnects to be subsequently formed. Patterning of the pMUT device structure 100 may be done using conventional photolithographic and etching techniques. By way of example, the conductive film 66 may be of metals such as Cr/Au, Ti/Au, Ti/Pt, Au, Ag, Cu, Ni, Al, Pt, In, Ir, InO₂, RuO₂, In₂O₃:SnO₂ (ITO) and (La,Sr)CoO₃ (LSCO), with respect to the bottom electrode 20, top electrode 32, and conformal conductive layer 42.

The SOI-pMUT device structure 100 is further processed to form the second dielectric film 28 and top electrode 32. Through-wafer vias 34 are formed, for example by deep reactive ion etching (DRIE). The conformal insulating layer 36, and conformal conductive film 42, are formed in the through-wafer vias as illustrated in FIG. 6. Electrical contact between the conductive film 66 and the conformal conductive film 42 provide a through-wafer interconnect. The SOI-pMUT device structure 100 is connected to a semiconductor device 44, such as through solder bumps 46, as shown in FIG. 6, to form a vertically integrated pMUT device 110. In other embodiments, the semiconductor device 44 may be electrically connected to the conformal conductive film 42 through metal contacts formed in an epoxy layer deposited on the surface of the semiconductor device which attaches the pMUT device to the semiconductor device, as previously described.

Application of the above method of enhancing receive signal may be integrated with an improved silicon-on-insulator (SOI) substrate pMUT device and/or vertically stacked ASIC devices as follows.

Previously described pMUT devices with air-backed cavities provided the bottom electrode in direct contact with the conformal metal layer in the air-backed cavity, or a metallized plug through an SOI layer to contact the plug metal to the conformal metal layer. The fabrication of an improved SOI air-backed cavity pMUT provides SiO₂ or device silicon structural layers as the membrane which may provide for more accurately targeting a specific resonance frequency, since frequency is dependent on membrane thickness and provides for direct electrical contact with the piezoelectric element through the air-back cavity. Thus, a heavily doped, electrically conductive, device silicon layer in the SOI substrate providing electrical interconnection between the bottom electrode and conformal metal layer through the air-backed cavity was envisaged. A pMUT of this embodiment is exemplified below with reference to FIG. 7.

SOI substrate 120 with a heavily doped (<0.1 ohm-cm resistivity) device silicon layer 162 is provided on the buried oxide layer 164 on the front surface of the substrate 120. A SiO₂ passivation layer 175 is thermally grown on the surface of device silicon layer 162 to prevent diffusion of bottom electrode layer 116 into doped device silicon layer 162 in subsequent processing steps. SiO₂ layer 175 is patterned by photolithography and etching. Bottom electrode layer 116 may be deposited by sputtering or electron beam evaporation and may be Pt or Pt/Ti. Ti may be used for adhesion of the Pt to SiO₂ layer. Preferably, the metal of bottom electrode 116 is able to withstand piezoelectric material anneal temperatures. The bottom electrode may be patterned by photolithography and etch or liftoff processing. The bottom electrode may be as described above.

Patterned piezoelectric elements 22 may be formed by depositing piezoelectric material by spin coating, sputtering, laser ablation or CVD, and annealing, typically at a temperature of 700° C. Patterning may be performed, for example, by photolithography and etching. Patterned piezoelectric elements 22 are etched such that the piezoelectric layer width is less than the width of the bottom electrode. This provides access to the bottom electrode such that the subsequent metal connector may be formed.

Metal connector layer 180 is deposited and patterned by photolithography and etch or liftoff processing. The metal connector layer 180 may be Ti/Pt, Ti/Au, or other metal as described above. Ti may be used for adhesion of Pt or Au to heavily doped device silicon layer 162. Metal connector layer 180 provides electrical contact between the bottom electrode 116 and heavily doped device silicon layer 162.

Device silicon layer 162 is patterned by photolithography and etched to provide an isolation trench 130 adjacent to each piezoelectric element 22 providing electrical isolation of the piezoelectric elements 22 within an array with respect to each other. Isolation trench 130 is etched to the buried SiO₂ layer 164.

Polymer dielectric layer 128 is deposited on top of piezoelectric elements 22 including trenches 130 and patterned by spin coating, photolithography and etching. Photoimageable polymeric dielectric materials may be used for the polymer dielectric layer 128. Polymer dielectric material may be polyimide, parylene, polydimethylsiloxane (PDMS), polytetrafluoroethylene (PTFE), polybenzocyclobutene (BCB) or other suitable polymers.

Metal ground plane layer 132 is deposited, for example by electron beam evaporation, sputtering or electroplating. Ti/Au or Ti/Cu may be used for metal ground plane layer 132.

Polymer passivation layer 190 is deposited, for example, by vapor deposition or spin coating. Polymer passivation layer 190 provides electrical and chemical insulation from fluid that may come in contact with the device surface during use (e.g., blood, water, silicone gel), and may also serves as an acoustic matching layer providing a lower acoustic impedance layer between the transducer face and the fluid.

Etching of back side of silicon substrate 120 provides air-backed cavities 150. Ground vias 131 are etched providing connection of the conformal conductor 143 to the doped silicon layer 162 and to the metal ground plane layer 132. Etching may be by deep reactive ion etching (DRIE).

Conformal insulator layer 136 is deposited on sidewalls 137 and base 125 of the air-backed cavities 150 as well as on back surface 111 of substrate 120. Conformal insulator layer 136 of base 125 is etched if a via is required, for example, for interconnection. Conformal insulator layer 136 may be polymer, oxide or nitride material.

Conformal metal layer 142 is deposited inside of air-backed cavity 150 including sidewalls 137 and base 125 and back surface 111 of substrate 120. Conformal metal layer 142 may be sputtered, e-beam evaporated, or CVD deposited.

Conformal metal layer 142 is patterned on back surface 111 of substrate 120 by photolithography and etched to electrically isolate piezoelectric elements 22 and ground via 131 from one another. Conformal metal layer 142 also provides interconnect pad 143 for electrical connection of the pMUT device to an IC device. Thus, electric contact from the piezoelectric element through the air-backed cavity of a SOI-pMUT device is provided with possible processing advantages and performance benefits.

In certain embodiments, application of the above method of generating an enhanced receive signal may be carried out using the pMUT devices or pMUT devices fabricated with a SOI substrate bonded to ASIC devices. Such vertically integrated devices include those previously described in co-pending U.S. patent application Ser. No. 11/068,776. An improved bonding structure for providing compactness of the pMUT-ASIC stack for application in imaging probes such as catheters of small diameter, for example, is as follows.

A pMUT substrate may be mechanically attached and electrically connected to an IC substrate such as an ASIC device, for example, as shown in FIG. 3. Connection of the pMUT to the IC substrate may be by epoxy bonding or by solder bump bonding. IC substrates bonded by solder bumps typically have thicknesses of multiple millimeters, depending on the number of IC layers. It is desirable to further reduce overall thickness and increase compactness of the pMUT-IC assembly. A preferred method for bonding of the pMUT and IC substrates is epoxy bonding. Epoxy bonding may provide greater physical compactness and lower overall thickness in the assembled device and may provide lower temperature processing steps compared to solder bumping.

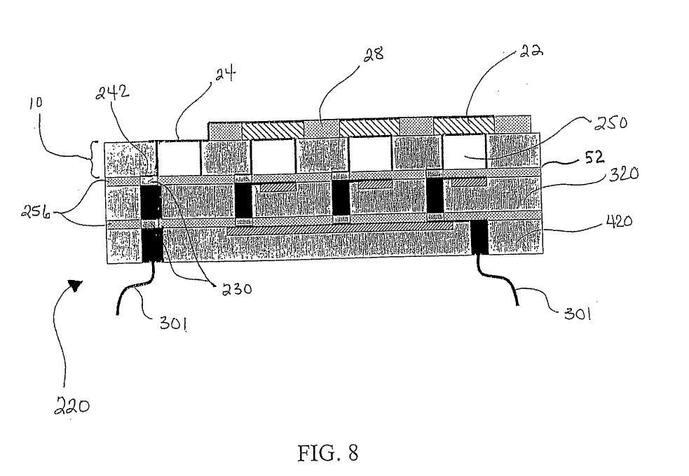

An example of an improved epoxy bonded pMUT-IC stack 220 is shown in FIG. 8. Epoxy interconnect layer 256 is deposited on surface of IC substrate 320 providing bonding with pMUT device 10. A conformal dielectric 52 is deposited to isolate the through-wafer electrical interconnects 230 and IC substrate 320. Through-wafer interconnects 230 may be etched in the IC layer and through the epoxy interconnect layer 256 to expose metal interconnection pad 242 on the back side of the pMUT device 10. The etching may be by DRIB, and the through-wafer interconnects 230 may be metallized using CVD and/or electroplating. A second IC substrate 420 may be subsequently bonded with vias formed similarly and electrical connections formed similarly. Electrical leads 301 (e.g., wires, flex cables, etc.) may be attached to the back side or one or more IC substrates to provide electrical connection from the pMUT-IC stack to the system electronics or catheter electrical connector.

The IC substrates may be thinned by chemical-mechanical polishing (CMP). Thinning of the IC silicon substrates using CMP may significantly reduce the overall thickness of the stack, and may provide a thickness of less than 1 mm for the entire stack. CMP may also provide for via etches that may be shallower and for via sizes that may be smaller, as aspect ratios of typically no more than 10:1 may be formed using conventional silicon etch and CVD metal via forming processes. The pMUT substrate may also be thinned by CMP or other process prior to forming air backed cavities 250.

Solder bump or wire bond stacking (e.g., system-on-chip or system-on-package) requires additional lateral area due to die handling and wirebonding constraints. The epoxy bonding method requires no additional lateral area, as fiducials may be formed on the back sides of the IC substrates, and alignment and bonding of two substrates may be formed by precision aligner-bonder equipment. Thus, when vias are etched in the silicon substrates, the vias are pre-aligned to the interconnect pads of the previous substrate. Therefore, the entire pMUT-IC stack 220 need be no larger in lateral area than the pMUT array itself.

pMUTs formed with through-wafer interconnects combined with control circuitry as described above thereby forming a transducer device may be further assembled into a housing assembly including external cabling to form an ultrasonic probe, such as an ultrasound imaging probe. The integration of pMUTs with control circuitry may significantly reduce the cabling required in the ultrasonic probe. The ultrasonic probe may also include various acoustic lens materials, matching layers, backing layers, and dematching layers. The housing assembly may form an ultrasonic probe for external ultrasound imaging, or a catheter probe for in vivo imaging. The shape of the ultrasound catheter probe housing may be of any shape such as rectangular, substantially circular or completely circular. The housing of the ultrasound catheter probe may be made from any suitable material such as metal, non-metal, inert plastic or like resinous material. For example, the housing may include a biocompatible material comprised of a polyolefin, thermoplastic, thermoplastic elastomer, thermoset or engineering thermoplastic or combinations, copolymers or blends thereof.

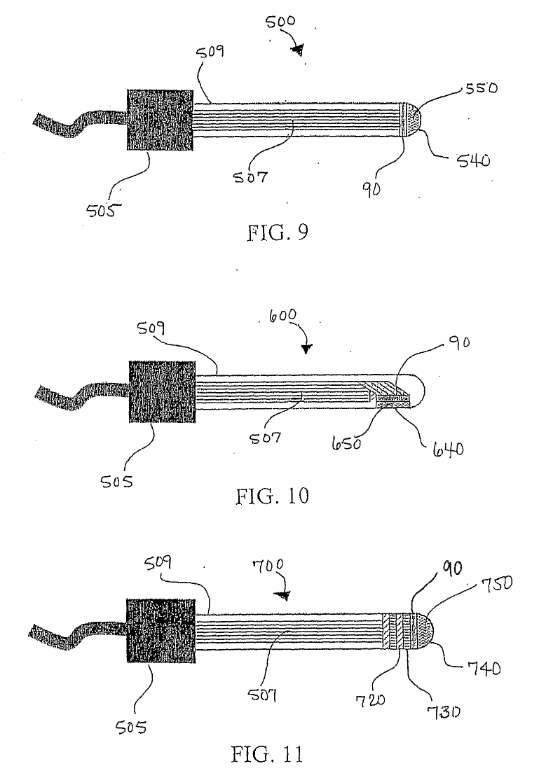

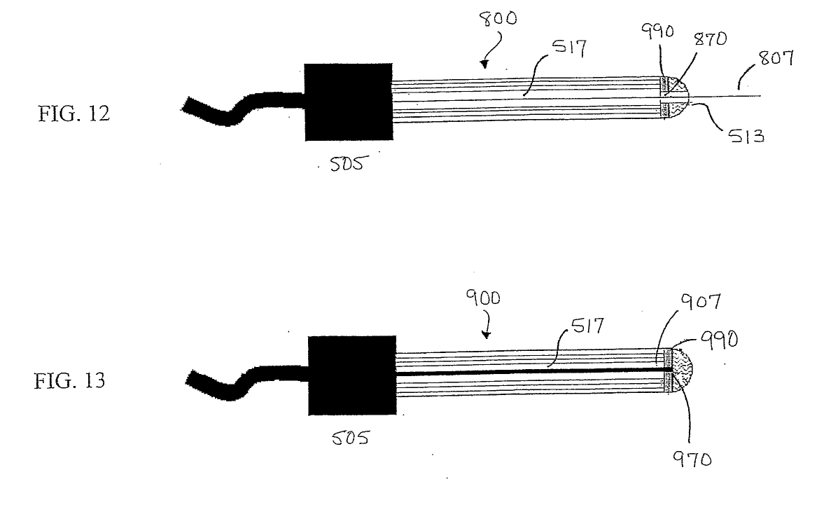

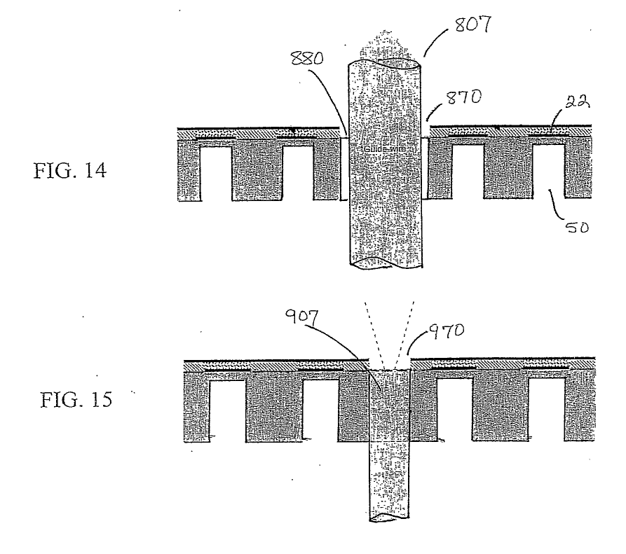

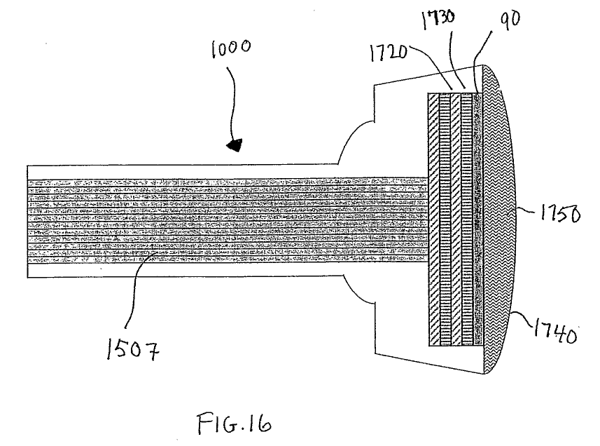

Methods for generating enhanced receive signal of an ultrasound catheter probe are provided. The methods comprise providing an ultrasound catheter probe comprising a pMUT or a pMUT integrated with an application-specific integrated circuit (ASIC) device assembly and incorporating the assembly in an imaging device and supplying a DC bias during receive flexural resonance mode of the pMUT for generating an enhanced receive signal from the pMUT. Such embodiments are further described with reference to FIGS. 9-15.

pMUT device 90 may be bonded to a flex cable 507 or other flexible wire connection providing imaging catheter devices 500, 600 as shown in FIGS. 9-10. This may be done by solder bump bonding, epoxy (conductive epoxy or combination of conductive and nonconductive epoxy), z-axis elastomeric interconnects, or other interconnection techniques used for catheter-based ultrasound transducers.

Referring to FIG. 9, forward viewing imaging catheter device 500 includes associated pMUT 90 integrated with flex cable 507 for imaging through an acoustic window 540. Side viewing catheter 600 includes associated pMUT 90 integrated with flex cable 507 and acoustic window 640, as depicted in FIG. 10. Catheters 500 and 600 include acoustically matching material 550, 650, respectively, directly in contact with pMUT 90. Acoustic matching material 550, 650 may be a low elastic modulus polymer, water or silicone gel.

Catheter 700 includes pMUT 90 with vertically integrated ASIC devices 720, 730 which may be multiplexer, amplifier or signal conditioning ASIC devices or combinations thereof. Additional ASIC devices may also be included such as high voltage drivers, beam formers or timing circuitry. Acoustic window 740 may include acoustically matching material 750 directly in contact with pMUT 90.

Imaging catheter devices 500, 600, 700 may have an outside diameter in the range of 3 French to 6 French (1-2 mm), but may also be as large as 12 French (i.e. 4 mm) for certain applications. Such a device may be able to access small coronary arteries. It is desired that minimal number of electrical wires be assembled in small catheter probes, thus miniature integrated circuit switches (e.g. multiplexers) can provide for reduction of electrical wires inside of the catheter. The housing 509 of imaging catheter device 500, 600, 700 may be highly flexible and may be advanced on a guide-wire, for example, in the epicardial coronary arteries.

Signal wires or flex cable leads can be connected directly with the through-wafer interconnects on the back side of the pMUT substrate as shown in FIG. 9. The wires or flex cable can be routed through the catheter body and connected through the I/O connector at the back end of the catheter to external control circuitry. However, it would be beneficial to reduce the number of electrical leads contained in the catheter sheath in order to allow greatest mechanical flexibility for steering/guiding the catheter through vessels. For example, a 7F (3 mm diameter) catheter, a 20×20 element pMUT array may be used to produce high quality images. In this case, a minimum of 1 wire per element totaling at least 400 wires would be required to drive the pMUT array at the tip of the catheter. This would leave little room for a guide wire to direct the catheter movement and little flexibility to bend the catheter.

Thus, in order to reduce the number of signal leads and signal noise in the catheter, the pMUT device may be integrated with control circuitry in the catheter tip. For example, as shown in FIG. 8, the readout function may be directly integrated with the transducer array using through-wafer interconnects. An amplifier ASIC may be bonded to the pMUT substrate and connected to the through-wafer interconnects of each pMUT element such that the ultrasonic signal received by each pMUT element is amplified independently to maximize signal-to-noise ratio. This direct integration may also greatly reduce the electrical lead length between the pMUT element and the amplifier to further reduce signal noise. By integrating a second multiplexing ASIC, the signal received by each transducer and sent to each amplifier may be multiplexed through a reduced number of signal wires to the I/O connector at the back end of the catheter. Thus, less wires are required within the catheter sheath. The speed of the multiplexing will determine the reduced number of signal wires that may be achieved. Reducing the number of leads also reduces the crosstalk between elements.

Through-wafer interconnects may be formed by etching the silicon substrate of the ASIC, coating the etched holes with conformal dielectric and metal layers, and plating metal to produce filled conductive vias, as described above. Multiple circuits may be stacked by epoxy bonding with aligned through-wafer interconnects.

In addition to integrating the receive function of the transducer array, the drive or transmit function may be integrated with the pMUT substrate in a similar fashion. High voltage drivers included in the ASIC stack may be used to generate the necessary to drive the transducer elements, and multiplexing circuitry can be used to address individual pMUT elements. Thus, 2D phased array operation may be achieved by multiplexing the drive signal with appropriate timing. At least one advantage of directly integrating the transmit function is that high voltage is generated directly adjacent to the pMUT array. High-voltage signals transmitted through the body of the catheter would be reduced or eliminated, thus improving the electrical safety of the catheter. Low voltage signals (3-5V) may be sent from the I/O connector to the integrated multiplexing and high voltage driver circuitry, and the drivers generate the higher transmit voltage through charge pumps and/or inductive transformers.

Other circuitry may be integrated in the ASIC stack such as timing and/or beam forming circuitry to control the transmit/receive signals and produce the ultrasound imaging signal from the raw pMUT signals. This integration may reduce the amount and size of electronics required in the external control unit, enabling a smaller, hand-held ultrasound imaging system or portable catheter-based ultrasound imaging system.

It is envisioned that the embodiments herein described are applicable to forward or side viewing catheters operating with 2D, 1.5D or 1D arrays.

Referring now to FIGS. 12-15, pMUT device 990 of catheters 800, 900 is constructed to provide for a manipulative member 807 or optical fiber 907. The manipulative member may be a catheter guide wire. The manipulative member may include a surgical instrument such as a scalpel, needle or syringe. The manipulative member may be remotely controlled through the catheter or housing assembly. Manipulative member 807 or optical fiber 907 is positioned in bore hole 870, 970, respectively. The manipulative means may be controlled externally. Bore 970 may include seal 880 to secure manipulative member 807 and to prevent seepage of fluid into the catheter. Manipulative member 807 may also be movable or retractable with respect to bore 870 and seal 880. Optical fiber 907 can be affixed directly to sidewalls of bore 970 and sealed with epoxy or other seal or adhesive. Such manipulative means such as guide wire, surgical tool or optical fiber may be adapted to stacked pMUT-IC devices in a similar manner. Bore holes 870, 970 may be provided during processing of the pMUT or pMUT-IC stack using etching processes, for example DRIE. The bore hole is cooperatively aligned with a suitable sized opening 513 in the distal end of the catheter. An internal passage 517 through the interior of the catheter housing communicable with the bore and opening 513 provides for the insertion and manipulation of the manipulative member.

The imaging catheter devices 600, 700, 800, 900 further comprise a steering mechanism 505 coupled to the proximal portion of the conduit. By way of example, at least one steering mechanism is disclosed in U.S. Pat. No. 6,464,645, which is incorporated by reference herein. A controller for the ultrasonic transducer assembly may also be provided that is contoured to a human hand to provide a comfortable and efficient one-handed operation of controls on the controller.

The catheter probes and pMUT transducer elements herein disclosed may be adapted to sterilization as is conventionally performed for medical devices. The pMUT devices and methods of generating enhanced receive signal described herein may be used for procedures such as real time, three-dimensional intracardiac or intravascular imaging, imaging for minimally invasive or robotic surgeries, catheter-based imaging, portable ultrasound probes, and miniature hydrophones. The pMUTs may optimized for operation in the frequency range of about 1-20 MHz.

The ultrasound catheter probe herein disclosed may be particularly suited for IVUS and ICE of coronary thrombosis of a coronary artery. Such treatment may be necessary to treat or possibly reduce coronary artery disease, atherosclerosis or other vascular related disorders.

The methods and embodiments herein described may be used to produce an external ultrasound probe with enhanced sensitivity. Thus, the vertically integrated pMUT device may also be adapted for use in external ultrasound probes, for example for cardiology, obstetrics, vascular, or urological imaging. Thus, as shown in FIG. 16, forward viewing imaging probe device 1000 includes associated pMUT 90 integrated with flex cable 1507 for imaging through an acoustic window 1740. Probe 1000 includes vertically integrated ASIC devices 1720, 1730, which may be multiplexer, amplifier or signal conditioning ASIC devices or combinations thereof with pMUT 90. Additional ASIC devices may also be included such as high voltage drivers, beam formers or timing circuitry. Acoustic window 1740 may include acoustically matching material 1750 directly in contact with pMUT 90.

pMUT arrays with 1D, 1.5D or 2D geometries can be fabricated and integrated with ASIC devices to provide electronic signal processing in the handle of the transducer probe. The pMUT-IC stack can be mounted in an external probe housing with an acoustic matching layer consisting of a low elastic modulus polymer, water, or silicone gel between the pMUT face and the housing wall. The pMUT-IC stack may be mounted to flex cable, ribbon cable, or standard signal wires for interface to the imaging system electronics.

Conventional ultrasound transducer arrays with integrated electronics for external ultrasound probes require costly, complex manufacturing techniques. An external pMUT-based probe may provide a lower cost, more manufacturable product due to semiconductor batch fabrication and integration techniques.

### Example

The method of generating enhanced receive signals from an ultrasound piezoelectric transducer are further described with reference to the following example.

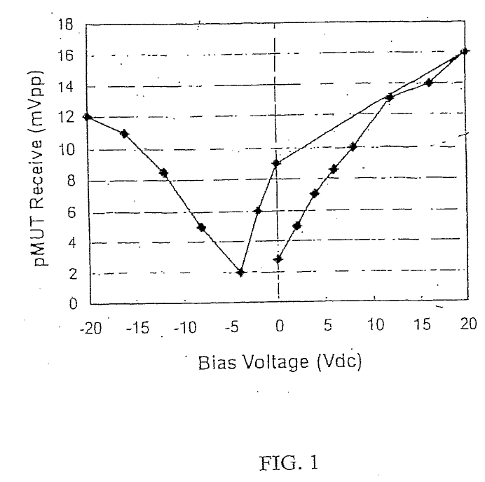

A single pMUT element was subjected to an DC bias from −20 Vdc to +20 Vdc. An acoustic signal provided by a separate piston transducer was aimed at the pMUT element. Signal received by the pMUT element was measured as a function of applied DC bias. Referring to FIG. 1, a graph depicting receive signal in millivolts peak-to-peak verses bias voltage is shown. The data in FIG. 1 represents the output response of the pMUT element for different levels of DC bias voltage. The DC bias voltage was varied from 0 V to +20V, back to 0V, then from 0V to −20V. Receive signal (mV) was recorded at each DC bias increment. FIG. 1 depicts the optimum DC bias voltages for increasing receive sensitivity with respect to the coercive field level in this particular piezoelectric thin film. When the DC bias was near the coercive voltage (approximately −5V) of the piezoelectric film in the pMUT element, receive sensitivity decreased. As the applied voltage increased the output signal of the pMUT element increased. Thus, the method of applying a DC bias to generate enhanced receive signal of a pMUT element was demonstrated. Optimization of the enhancement in receive signal may be obtained by adjusting the DC bias while monitoring the receive signal from a piezoelectric membrane of known thickness.

While the invention has been described in detail and with reference to specific embodiments thereof, it will be apparent to one skilled in the art that various changes and modifications may be made without departing from the spirit and scope of the invention.

What is claimed is:

1. A method of generating an enhanced receive signal from a piezoelectric ultrasound transducer, the method comprising:
providing a piezoelectric ultrasound transducer, the piezoelectric ultrasound transducer comprising a piezoelectric element operable in flexural mode;
receiving acoustic energy by the piezoelectric element, the acoustic energy convertible to an electrical voltage by flexural mode resonance of the piezoelectric element;
applying a DC bias to the piezoelectric element prior to receiving the acoustic signal and/or concurrently with receiving the acoustic energy; and
generating an enhanced receive signal from the piezoelectric transducer by converting the received acoustic energy to an electrical voltage by flexural mode resonance of the piezoelectric element;
wherein the enhanced receive signal generated by the piezoelectric transducer is greater than a receive signal generated by the piezoelectric transducer in the absence of applying a DC bias.

2. The method of claim 1, wherein the DC bias is applied during the flexural mode resonance of the piezoelectric element.

3. The method of claim 1, wherein the DC bias is applied before the acoustic signal reaches the transducer and during the flexural mode resonance of the piezoelectric element.

4. The method of claim 1, wherein the DC bias is applied before the acoustic signal reaches the transducer and is terminated during the flexural mode resonance of the piezoelectric element.

5. The method of claim 1, wherein the applied DC bias is maintained during the flexural mode resonance of the piezoelectric element.

6. The method of claim 1, further comprising applying signal conditioning to the enhanced receive signal.

7. The method of claim 6, wherein the signal conditioning separates the DC bias signal from the generated enhanced receive signal.

8. The method of claim 6, wherein the signal conditioning amplifies the enhanced receive signal.

9. A method of generating an enhanced receive signal from a piezoelectric ultrasound transducer, the method comprising:
providing a piezoelectric ultrasound transducer, the piezoelectric ultrasound transducer comprising a piezoelectric element operable in flexural mode;
applying a sine wave bipolar transmit cycle pulse to the piezoelectric element to produce an acoustic signal providing an acoustic echo, the sine wave bipolar transmit cycle pulse having a maximum peak voltage;
receiving the acoustic echo by the piezoelectric element, the acoustic echo convertible to an electrical voltage by flexural mode resonance of the piezoelectric element;
applying a DC bias to the piezoelectric element prior to receiving the acoustic echo and/or concurrently with receiving the acoustic echo; and
generating an enhanced receive signal from the piezoelectric transducer by converting the received acoustic echo to an electrical voltage by flexural mode resonance of the piezoelectric element;
wherein the enhanced receive signal generated by the piezoelectric transducer is greater than a receive signal generated by the piezoelectric transducer in the absence of applying a DC bias.

10. The method of claim 9, wherein the DC bias is applied during the flexural mode resonance of the piezoelectric element.

11. The method of claim 9, wherein the DC bias is applied before the acoustic echo reaches the transducer and during the flexural mode resonance of the piezoelectric element.

12. The method of claim 9, wherein the DC bias is applied before the acoustic signal reaches the transducer and is terminated during the flexural mode resonance of the piezoelectric element.

13. The method of claim 9, wherein the applied DC bias is maintained during the flexural mode resonance of the piezoelectric element.

14. The method of claim 9, wherein the DC bias is of opposite polarity to that of the maximum peak voltage of the sine wave bipolar transmit cycle pulse.

15. The method of claim 9, further comprising applying signal conditioning to the enhanced receive signal.

16. The method of claim 15, wherein the signal conditioning separates the DC bias signal from the generated enhanced receive signal.

17. The method of claim 15, wherein the signal conditioning amplifies the enhanced receive signal.

18. The method of claim 1, wherein the piezoelectric ultrasound transducer comprises:
a substrate;
sidewalls defining an opening through the substrate;
a bottom electrode on the substrate spanning the opening;
a piezoelectric element on the bottom electrode; and
a conformal conductive film on the sidewall of the opening in contact with the bottom electrode through the substrate, wherein an open cavity is maintained in the opening.

19. The method of claim 18, further comprising a conformal insulating film on the sidewall of the opening underlying the conformal conductive film.

20. The method of claim 18, further comprising a first dielectric film on the substrate underlying the bottom electrode.

21. The method of claim 18, further comprising a second dielectric film surrounding the piezoelectric element, wherein top edges of the piezoelectric element are covered with the second dielectric film.

22. The method of claim 18, further comprising a top electrode in contact with the piezoelectric element.

23. The method of claim 18, wherein the piezoelectric transducer is a pMUT.

24. The method of claim 18, further comprising spaced-apart vias through the first dielectric and through a portion of the substrate.

25. The method of claim 18, wherein the substrate comprises a silicon wafer.

26. The method of claim 18, wherein said silicon wafer is a silicon-on-insulator wafer.

27. The method of claim 26, further comprising a doped silicon layer in electrical contact between the bottom electrode of the piezoelectric elements and the conformal conductive film of the opening.

28. The method of claim 18, wherein the piezoelectric ultrasound transducer further comprises a vertically integrated semiconductor device attached to the ultrasonic transducer of claim 18 wherein the conformal conductive film is electrically connected to the semiconductor device.

29. The method of claim 1, wherein the piezoelectric ultrasound transducer comprises:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate, wherein each pair of spaced-apart bottom electrodes are in contact with each of the spaced-apart piezoelectric elements;
a conformal conductive film on each of the sidewalls of the plurality of openings, each conformal conductive film in electrical interconnection with the bottom electrode through the substrate, wherein open cavities are maintained in each of the openings.

30. The method of claim 29, wherein the piezoelectric ultrasound transducer is a pMUT.

31. The method of claim 29, wherein the substrate comprises a silicon wafer.

32. The method of claim 31, wherein said silicon wafer is a silicon-on-insulator wafer.

33. The method of claim 32, further comprising a doped silicon layer in electrical contact between the bottom electrode of the piezoelectric elements and the conformal conductive film of the opening.

34. The method of claim 29, wherein the piezoelectric ultrasound transducer further comprises a vertically integrated semiconductor device attached to the ultrasonic transducer of claim 29 wherein the conformal conductive film is electrically connected to the semiconductor device.

35. An ultrasound imaging catheter comprising:
a housing having a distal end for insertion into and manipulation within a vascularized organism and a proximal end for providing a user with control over the manipulation of the distal end of the catheter within the vascularized organism; and
a piezoelectric ultrasound transducer positioned within the housing proximal to the distal end of the housing, the transducer comprising:
a substrate;
a plurality sidewalls defining a plurality of openings through the substrate;
spaced-apart bottom electrodes on the substrate, wherein each spaced-apart bottom electrode spans one of the plurality of openings;
spaced-apart piezoelectric elements on each of the bottom electrodes;
a conformal conductive film on each of the sidewalls of the plurality of openings, each conformal conductive film in contact with the bottom electrodes through the substrate, wherein open cavities are maintained in each of the openings.

36. The ultrasound imaging catheter of claim 35, wherein the piezoelectric ultrasound transducer is a pMUT.

37. The ultrasound imaging catheter of claim 35, further comprising means for applying a DC bias to the piezoelectric transducer.

38. The ultrasound imaging catheter of claim 35, further comprising an acoustic window proximal to the distal end of the catheter housing and adjacent the piezoelectric ultrasound transducer.

39. The ultrasound imaging catheter of claim 38, further comprising a matching acoustic layer positioned between the acoustic window and in contact with the piezoelectric ultrasound transducer.

40. The ultrasound imaging catheter of claim 35, wherein the distal end of the catheter housing comprises an opening.

41. The ultrasound imaging catheter of claim 40, wherein the catheter housing further comprises an internal passage in communication with the opening at the distal end of the catheter housing.

42. The ultrasound imaging catheter of claim 41, wherein the substrate of the piezoelectric ultrasound transducer comprises a bore through the substrate, the bore communicable with the internal passage and the opening at the distal end of the catheter housing.

43. The ultrasound imaging catheter of claim 42, further comprising a manipulative member communicable with the internal passage, opening and bore.

44. The ultrasound imaging catheter of claim 43, wherein the manipulative member is a guide wire.

45. The ultrasound imaging catheter of claim 43, wherein the manipulative member is a surgical instrument or optical imaging fiber.

46. The ultrasound imaging catheter of claim 35, wherein the piezoelectric ultrasound transducer is configured for forward- or side-imaging.

47. The ultrasound imaging catheter of claim 35, further comprising a conformal insulating film on each of said sidewalls of the plurality of openings underlying said conformal conductive film.

48. The ultrasound imaging catheter of claim 35, further comprising a first dielectric film on said substrate underlying said bottom electrodes.

49. The ultrasound imaging catheter of claim 35, further comprising a second dielectric film between said piezoelectric elements.

50. The ultrasound imaging catheter of claim 49, wherein said second dielectric film is disposed on top edges of said piezoelectric elements.

51. The ultrasound imaging catheter of claim 35, further comprising a ground pad on said substrate.

52. The ultrasound imaging catheter of claim 51, further comprising a top electrode in contact with said piezoelectric elements and said ground pad.

53. The ultrasound imaging catheter of claim 52, wherein said top electrode and said conformal conductive film comprise a metal film.

54. The ultrasound imaging catheter of claim 35, wherein the piezoelectric elements form a one-dimensional or two-dimensional array.

55. The ultrasound imaging catheter of claim 35, wherein the substrate comprises a silicon wafer.

56. The ultrasound imaging catheter of claim 53, wherein said silicon wafer is a silicon-on-insulator wafer.

57. The ultrasound imaging catheter of claim 56, further comprising a doped silicon layer in electrical contact between the bottom electrode of the piezoelectric elements and the conformal conductive film of the opening.

58. The ultrasound imaging catheter of claim 35, further comprising the piezoelectric ultrasonic transducer vertically integrated to a semiconductor device, the transducer being attached to and electrically connected to the semiconductor device.

59. The ultrasound imaging catheter of claim 58, wherein said semiconductor device is a complementary metal oxide semiconductor chip.

60. The ultrasound imaging catheter of claim 58, wherein the semiconductor device provides means for applying a DC bias to the piezoelectric transducer.

61. The ultrasound imaging catheter of claim 58, further comprising a polymer film on a surface of the semiconductor device facing the open cavities.

62. The ultrasound imaging catheter of claim 58, further comprising an adhesive layer between said ultrasonic transducer and said semiconductor device.

63. The ultrasound imaging catheter of claim 62, further comprising metal contacts in said adhesive layer electrically connecting said ultrasonic transducer to said semiconductor device.

64. The ultrasound imaging catheter of claim 63, wherein the metal contacts are vias etched through the adhesive layer between said ultrasonic transducer and said semiconductor device.

65. The ultrasound imaging catheter of claim 35, wherein each of the plurality of piezoelectric elements may be operated independently, all elements may be operated simultaneously, or subsets of elements may be electrically connected to form larger independently operated subsets of elements in an array.

66. An ultrasound imaging catheter comprising:
a housing having a distal end for insertion into and manipulation within a vascularized organism and a proximal end for providing a user with control over the manipulation of the distal end of the catheter within the vascularized organism; and
a piezoelectric ultrasound transducer positioned within the housing proximal to the distal end, the transducer comprising:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate and each pair of spaced-apart bottom electrodes are in contact with each of the spaced-apart piezoelectric elements;
a conformal conductive film on each of the sidewalls of the plurality of openings, each conformal conductive film in electrical interconnection with the bottom electrodes through the substrate, wherein open cavities are maintained in each of the openings;
a ground pad on the substrate;
a second dielectric film between the piezoelectric elements;
a top electrode in contact with the piezoelectric elements and the ground pad; and
a semiconductor device attached to the ultrasonic transducer, wherein the conformal conductive film is electrically connected to the semiconductor device.

67. The ultrasound imaging catheter of claim 66, wherein the piezoelectric ultrasound transducer is a pMUT.

68. The ultrasound imaging catheter of claim 66, further comprising means for applying a DC bias to the piezoelectric transducer.

69. The ultrasound imaging catheter of claim 68, wherein the means for applying a DC bias to the piezoelectric transducer are integrated into the semiconductor device.

70. The ultrasound imaging catheter of claim 66, further comprising an acoustic window proximal to the distal end of the catheter housing and adjacent the piezoelectric ultrasound transducer.

71. The ultrasound imaging catheter of claim 70, further comprising a matching acoustic layer positioned between the acoustic window and in contact with the piezoelectric ultrasound transducer.

72. The ultrasound imaging catheter of claim 66 wherein the distal end of the catheter housing comprises an opening.

73. The ultrasound imaging catheter of claim 72, wherein the catheter housing further comprises an internal passage in communication with the opening at the distal end of the catheter housing.

74. The ultrasound imaging catheter of claim 73, wherein the substrate of the piezoelectric ultrasound transducer comprises a bore through the substrate, the bore communicable with the internal passage and the opening at the distal end of the catheter housing.

75. The ultrasound imaging catheter of claim 74, further comprising a manipulative member communicable with the internal passage, opening and bore.

76. The ultrasound imaging catheter of claim 75, wherein the manipulative member is a guide wire.

77. The ultrasound imaging catheter of claim 75, wherein the manipulative member is a surgical instrument or optical imaging fiber.

78. The ultrasound imaging catheter of claim 66, wherein the piezoelectric ultrasound transducer is configured for forward- or side-imaging.

79. The ultrasound imaging catheter of claim 66, further comprising spaced-apart vias through said first dielectric and through a portion of said substrate.

80. The ultrasound imaging catheter of claim 79, further comprising metallization in said spaced-apart vias providing electrical contact between said bottom electrode and said conformal conductive film.

81. The ultrasound imaging catheter of claim 80, wherein the spaced-apart vias are etched through the adhesive layer between said ultrasonic transducer and said semiconductor device.

82. The ultrasound imaging catheter of claim 66, further comprising a polymer film on a surface of the semiconductor device facing the open cavities.

83. The ultrasound imaging catheter of claim 66, wherein said semiconductor device is a complementary metal oxide semiconductor chip.

84. The ultrasound imaging catheter of claim 66, wherein the substrate comprises a silicon wafer.

85. The ultrasound imaging catheter of claim 84, wherein said silicon wafer is a silicon-on-insulator wafer.

86. The ultrasound imaging catheter of claim 85, further comprising a doped silicon layer between the bottom electrode of the piezoelectric elements and the conformal conductive film of the opening.

87. The ultrasound imaging catheter of claim 66, further comprising an adhesive layer between said ultrasonic transducer and said semiconductor device.

88. The ultrasound imaging catheter of claim 87, further comprising metal contacts in said adhesive layer electrically connecting said ultrasonic transducer to said semiconductor device.

89. The ultrasound imaging catheter of claim 88, wherein the metal contacts are vias etched through the adhesive layer between said ultrasonic transducer and said semiconductor device.

90. The ultrasound imaging catheter of claim 66, wherein each of the plurality of piezoelectric elements may be operated independently, all elements may be operated simultaneously, or subsets of elements may be electrically connected to form larger independently operated subsets of elements in an array.

91. The ultrasound imaging catheter of claim 66, wherein the piezoelectric elements form a one-dimensional or two-dimensional array.

92. An ultrasound imaging probe comprising:
a housing having a distal end;
a piezoelectric ultrasound transducer positioned within the housing proximal to the distal end, the transducer comprising:
a substrate;
a plurality of sidewalls defining a plurality of openings through the substrate;
spaced-apart bottom electrodes on the substrate, wherein each spaced-apart bottom electrode spans one of the plurality of openings;
spaced-apart piezoelectric elements on each of the bottom electrodes; and
a conformal conductive film on each of the sidewalls of the plurality of openings, wherein each conformal conductive film is contact with one or more of the bottom electrodes and open cavities are maintained in each of the openings; and
means for applying a DC bias to the piezoelectric transducer.

93. An ultrasound imaging probe comprising:
a housing having a distal end;
a piezoelectric ultrasound transducer positioned within the housing proximal to the distal end, the transducer comprising:
a substrate;
a plurality of sidewalls defining a plurality of openings through the substrate;
a first dielectric layer on the substrate;
spaced-apart bottom electrodes on the first dielectric layer;
each spaced-apart bottom electrode spanning one of the plurality of openings;
spaced-apart piezoelectric elements on each of the bottom electrodes;
a conformal insulating film on each of the sidewalls of the plurality of openings;
a conformal conductive film on each of the conformal insulating films, wherein each conformal conductive film is in contact with one or more of the bottom electrodes and an open cavity is maintained in each of the openings;
a ground pad on the substrate;
a second dielectric film between the piezoelectric elements;
a top electrode in contact with the piezoelectric elements and the ground pad; and
a semiconductor device attached to the ultrasonic transducer, wherein the conformal conductive film is electrically connected to the semiconductor device; and
means for applying a DC bias to the piezoelectric transducer.

94. An ultrasound imaging probe comprising:
a housing having a distal end;
a piezoelectric ultrasound transducer positioned within the housing proximal to the distal end, the transducer comprising:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate, wherein each pair of spaced-apart bottom electrodes are in contact with each of the spaced-apart piezoelectric elements;
a conformal conductive film on each of the sidewalls of the plurality of openings, each conformal conductive film in electrical interconnection with the bottom electrodes through the substrate, wherein open cavities are maintained in each of the openings.

95. An ultrasound imaging probe comprising:
a housing having a distal end;
a piezoelectric ultrasound transducer positioned within the housing proximal to the distal end, the transducer comprising:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate, wherein each pair of spaced-apart bottom electrodes are in contact with each of the spaced-apart piezoelectric elements;
a conformal insulating film on each of the sidewalls of the plurality of openings;
a conformal conductive film on each of the sidewalls of the plurality of openings, each conformal conductive film in electrical interconnection with the bottom electrode through the substrate, wherein open cavities are maintained in each of the openings;
a ground pad on the substrate;
a second dielectric film between the piezoelectric elements;
a top electrode in contact with the piezoelectric elements and the ground pad; and
a semiconductor device attached to the ultrasonic transducer, wherein the conformal conductive film is electrically connected to the semiconductor device.

96. A piezoelectric ultrasound transducer comprising:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate, wherein each pair of spaced-apart bottom electrodes are in contact with each of the spaced-apart piezoelectric elements;
a conformal conductive film on each of the sidewalls of the plurality of openings, each conformal conductive film in electrical interconnection with the bottom electrodes through the substrate, wherein open cavities are maintained in each of the openings.

97. A piezoelectric ultrasound transducer comprising:
a substrate;
a plurality of sidewalls defining a plurality of openings partially through the substrate;
spaced-apart piezoelectric elements on the substrate, wherein each spaced-apart piezoelectric element is positioned over one of the plurality of openings;
pairs of spaced-apart bottom electrodes on the substrate, wherein each pair of spaced-apart bottom electrodes are in contact with each of the spaced-apart piezoelectric elements;
a conformal insulating film on each of the sidewalls of the plurality of openings;
a conformal conductive film on each of the sidewalls of the plurality of openings, each conformal conductive film in electrical interconnection with the bottom electrode through the substrate, wherein open cavities are maintained in each of the openings;
a ground pad on the substrate;
a second dielectric film between the piezoelectric elements;
a top electrode in contact with the piezoelectric elements and the ground pad; and
a semiconductor device attached to the ultrasonic transducer, wherein the conformal conductive film is electrically connected to the semiconductor device.

98. A method of generating enhanced receive signals of a flex mode transducer, the method comprising:
providing a piezoelectric ultrasound transducer, the piezoelectric ultrasound transducer comprising a piezoelectric element operable in flexural mode and having a ferroelectric coercive voltage;
applying a transmit voltage sine wave signal, wherein the transmit voltage sine wave signal is greater than the ferroelectric coercive voltage;
generating an acoustic signal as a result of the applied transmit voltage sine wave signal, the acoustic signal providing an acoustic echo;
receiving the acoustic echo by the piezoelectric element and converting the acoustic echo to an electrical voltage by flexural mode resonance of the piezoelectric element;
generating an enhanced receive signal wherein the enhanced receive signal generated by the piezoelectric transducer is greater than a receive signal generated by the piezoelectric transducer in the absence of a transmit voltage sine wave signal.

99. The method of claim 98, further comprising applying an additional half-wave transmit voltage sine wave signal, where the additional sine wave signal is greater than the ferroelectric coercive voltage.

100. The method of claim 98, further comprising:
applying a DC bias to the piezoelectric element prior to receiving the acoustic echo and/or concurrently with receiving the acoustic echo.
